(19) United States
(12) Patent Application Publication
(10) Pub. No.: US 2020/0188460 A1
(43) Pub. Date: Jun. 18, 2020

(54) PHARMACEUTICAL COMPOSITION FOR WOUND HEALING COMPRISING CERIPORIA LACERATA CULTURE MEDIUM

(71) Applicant: FUGENBIO CO., LTD., Seoul (KR)

(72) Inventors: Yoon Soo KIM, Seongnam-si (KR); Na Yeon KIM, Sejong (KR)

(73) Assignee: Fugenbio Co., Ltd., Seoul (KR)

(21) Appl. No.: 16/615,076

(22) PCT Filed: Jun. 29, 2018
(86) PCT No.: PCT/KR2018/007419

§ 371 Date: Nov. 19, 2019

(30) Foreign Application Priority Data
Jun. 30, 2017 (KR) 10-2017-0083249

(51) Int. Cl. A61K 36/07; A61P 17/02

(52) U.S. Cl. CPC A61K 36/07; A61K 2236/35; A61K 2236/33; A61P 17/02

(57) ABSTRACT

The present invention relates to a pharmaceutical composition for wound healing and a cosmetic composition for wound alleviation, each composition comprising a culture medium of Ceriporia lacerata mycelia or an extract thereof as an effective ingredient. A culture medium of Ceriporia lacerata mycelia or an extract thereof according to the present invention was found to increase cell proliferation rates of the murine myoblast C2C12. In addition, the mycelium culture medium or an extract thereof was identified to promote wound healing HaCaT cells, which are human keratinocytes. Therefore, the culture medium of Ceriporia lacerata mycelia or an extract thereof according to the present invention can be useful for healing skin wounds.

### TECHNICAL FIELD

The present invention relates to a pharmaceutical composition for wound healing comprising, as an active ingredient, a mycelial culture broth of Ceriporia lacerata or an extract thereof.

### BACKGROUND ART

The skin is the primary protective barrier of the human body, which protects organs in the body from the external environment, including temperature changes, humidity changes, ultraviolet light, harmful substances, and the like. Damage to the skin tissue is a wound, and if the wound is not properly treated, it may cause fatal damage to organs in the body due to bacterial infection or the like. When a wound occurs, it is important to treat the damaged skin site as soon as possible to be restored similar to the original skin structure.

Wound healing consists of three phases: the first phase of hemostasis and inflammation; the second phase of proliferation; and the third phase of remodeling, and each phase continuously proceeds in a concomitant manner. The stage where fibroblasts substantially migrate to a wound site to be recovered is a proliferative phase. In the proliferative phase, neovascularization, collagen accumulation, granulation tissue formation, epithelialization, wound contraction, and the like occur. In addition, in the proliferative phase, fibroblasts appear at a wound site as an early stage for wound fusion and rearrangement of collagenous fibers. Therefore, attempts have been made to find a substance that activates the proliferative phase, which is a stage where cellular tissues substantially migrate to the wound site.

Meanwhile, Ceriporia lacerata is known to be a type of white rot fungi, which performs co-metabolism called lignin decomposition to use carbon sources such as cellulose, hemicellulose, other polysaccharides, and glycerol in the ecosystem.

### DISCLOSURE

### Technical Problem

As a result of having conducted research on a substance capable of more efficiently healing skin wounds, the inventors of the present invention confirmed that a mycelial culture broth of Ceriporia lacerata activated cell proliferation and promoted wound healing, and thus completed the present invention.

### Technical Solution

In accordance with one aspect of the present invention, provided is a pharmaceutical composition for wound healing including, as an active ingredient, a mycelial culture broth of Ceriporia lacerata or an extract thereof.

In accordance with another aspect of the present invention, provided is a cosmetic composition for wound healing including, as an active ingredient, a mycelial culture broth of Ceriporia lacerata or an extract thereof.

In accordance with yet another aspect of the present invention, provided is a use of a mycelial culture broth of Ceriporia lacerata or an extract thereof for healing a skin wound.

In accordance with yet another aspect of the present invention, provided is a use of a mycelial culture broth of Ceriporia lacerata or an extract thereof for preparing a drug for healing a skin wound.

In accordance with yet another aspect of the present invention, provided is a method of healing a skin wound, including administering a mycelial culture broth of Ceriporia lacerata or an extract thereof to a subject in need thereof.

### Advantageous Effects

According to the present invention, it was confirmed that a mycelial culture broth of Ceriporia lacerata or an extract thereof increased the proliferation rate of C2C12 cells, which are mouse myoblasts. It was also confirmed that the mycelial culture broth of Ceriporia lacerata or an extract thereof promoted wound healing of HaCaT cells, which are human keratinocytes. Therefore, the mycelial culture broth of Ceriporia lacerata or an extract thereof, according to the present invention, can be effectively used to heal a skin wound.

### DESCRIPTION OF DRAWINGS

FIG. 1 is a set of images showing, after treatment of wounded HT-29 cells with a mycelial culture broth of Ceriporia lacerata, a wound size according to the concentration and culture time of the culture broth.

FIG. 2 is a graph showing, after treatment of wounded HT-29 cells with a mycelial culture broth of Ceriporia lacerata, a wound size according to the concentration and culture time of the culture broth.

FIG. 3 is a graph showing, after treatment of C2C12 cells with a mycelial culture broth of Ceriporia lacerata, a cell proliferation rate according to the concentration of the culture broth.

FIG. 4 is a set of images showing, after treatment of wounded HaCaT cells with a mycelial culture broth of Ceriporia lacerata, a wound size according to the concentration and culture time of the culture broth.

FIG. 5 is a graph showing, after treatment of HaCaT cells with a mycelial culture broth of Ceriporia lacerata, a cell proliferation rate according to the concentration of the culture broth.

### BEST MODE

An embodiment of the present invention provides a pharmaceutical composition for wound healing including, as an active ingredient, a mycelial culture broth of Ceriporia lacerata or an extract thereof.

The mycelial culture broth may be obtained by culturing a patent strain grown through sub-culturing of Ceriporia lacerata. The mycelial culture broth may be obtained through main fermentation or pre-fermentation before main fermentation. The production rate and culture termination time of the mycelium may vary depending on culture conditions, e.g., culture temperature and time. The culture termination time is not set to a specific time, but may be day 11, day 13, day 20, or day 25 from the culture starting date when cultured at a temperature of 15° C. to 30° C.

The mycelial culture broth may be obtained through culturing at a temperature of 15° C. to 30° C. for 3 days or longer. Specifically, the mycelial culture broth may be obtained through culturing at a temperature of 15° C. to 30° C. for 9 days or longer or 11 days or longer.

In addition, the mycelial culture broth may be obtained through culturing at a temperature of 20° C. to ° C. for 3 days or longer. Specifically, the mycelial culture broth may be obtained through culturing at a temperature of 20° C. to 25° C. for 9 days or longer or 11 days or longer.

In addition, the mycelial culture broth may be obtained through culturing at a temperature of 15° C. to 30° C. for 3 days to 25 days, 9 days to 13 days, or 9 days to days. Specifically, the mycelial culture broth may be obtained through culturing at a temperature of 20° C. to 25° C. for 3 days to 25 days, 9 days to 13 days, or 9 days to 11 days.

In one embodiment of the present invention, the mycelial culture broth may be obtained by culturing a mycelium at a temperature of 20° C. to 25° C. for 9 days or 11 days. The culture may be liquid culture.

A medium for culturing the Ceriporia lacerata mycelium may be a medium containing glucose. In addition, the medium may further include starch, soybean flour, an antifoaming agent, distilled water, and the like. The components of the medium and the contents thereof may be appropriately changed for sufficient growth of the mycelium. In one embodiment of the present invention, the medium for culture may include, per 1 L of the medium, 12.5 g of glucose, 2.5 g of starch, 5 g of soybean flour, and 0.125 g of an antifoaming agent.

In one embodiment of the present invention, the mycelial culture broth of Ceriporia lacerata may be dried and powdered. The drying process may be performed at a temperature of 40° C. or less or 30° C. or less for 48 hours to 96 hours to prevent the removal of the active material. For the drying process, it is preferable to use a vacuum freeze dryer rather than a vacuum dryer in which the evaporation temperature is set relatively high, so that a change in the content of the active material is minimized.

The pharmaceutical composition for wound healing may include an extract of a mycelial culture broth of Ceriporia lacerata, as an active ingredient. The extract may be prepared by extracting a mycelial culture broth of Ceriporia lacerata with a solvent. In addition, the extract may be prepared by drying and powdering a mycelial culture broth of Ceriporia lacerata and extracting the powdered mycelial culture broth with a solvent.

In one embodiment of the present invention, the solvent may be a solvent selected from the group consisting of distilled water, C₁ to C₄ lower alcohol, acetone, ether, chloroform, and ethyl acetate, or a mixture thereof. Specifically, the solvent may be a solvent selected from the group consisting of water, methanol, ethanol, butanol, acetone, and ethyl acetate, or a mixture thereof, preferably, ethyl acetate.

The inventors of the present invention conducted experiments using a human or mouse cell line to verify a wound healing effect of a mycelial culture broth of Ceriporia lacerata.

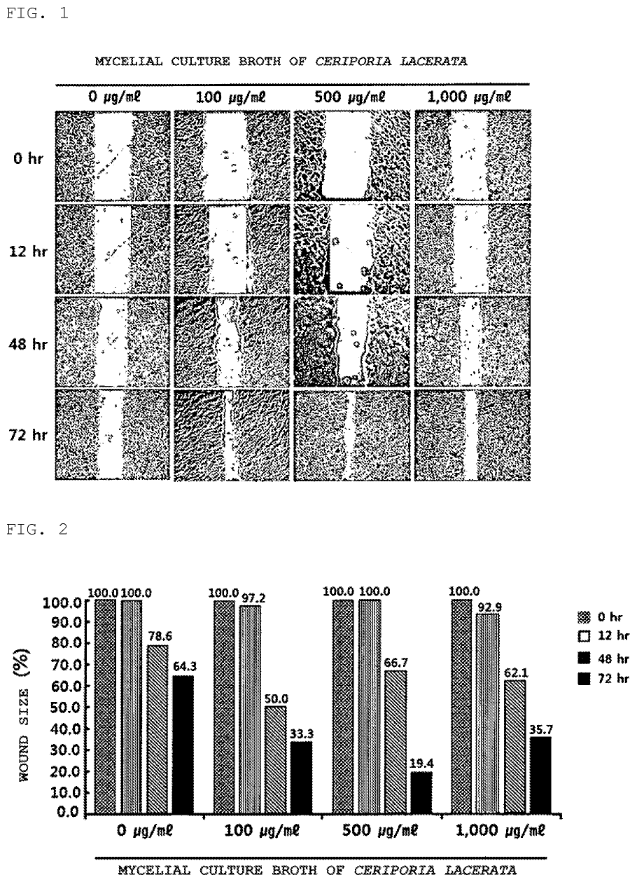

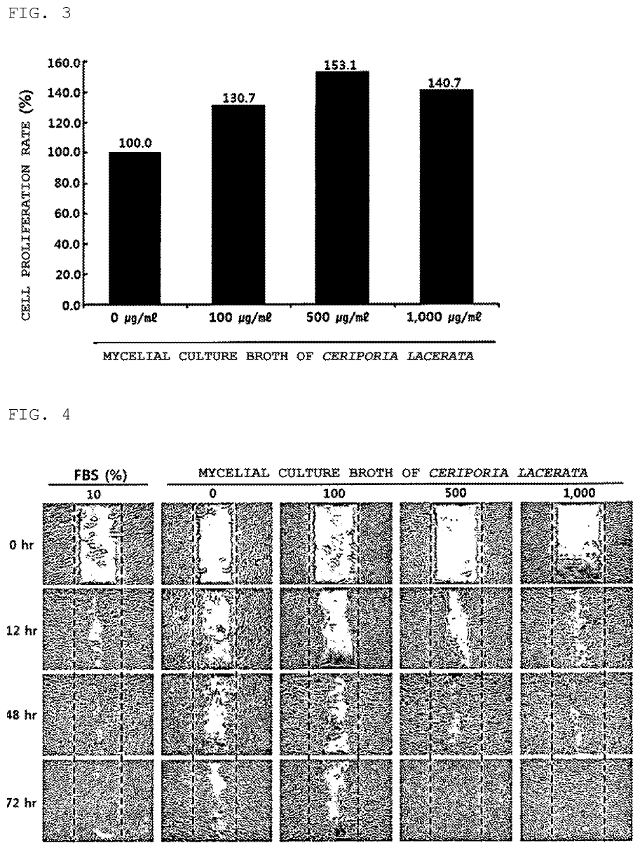

Specifically, HT-29 cells wounded at the center thereof were treated with a mycelial culture broth of Ceriporia lacerata, and then cultured for 12 hours, 48 hours, and 72 hours, and changes in wound size according to the concentration and culture time of the mycelial culture broth were observed. As a result, it was confirmed that the wound size was significantly reduced in groups treated with the mycelial culture broth of Ceriporia lacerata (see FIGS. 1 and 2).

In addition, C2C12 cells, which belong to a mouse myoblast cell line, were treated with the mycelial culture broth of Ceriporia lacerata, and then cultured for 12 hours, hours, and 72 hours, and a cell proliferation rate according to each culture time was measured. As a result, it was confirmed that the cell proliferation rate was increased in the groups treated with the mycelial culture broth of Ceriporia lacerata (see FIG. 3).

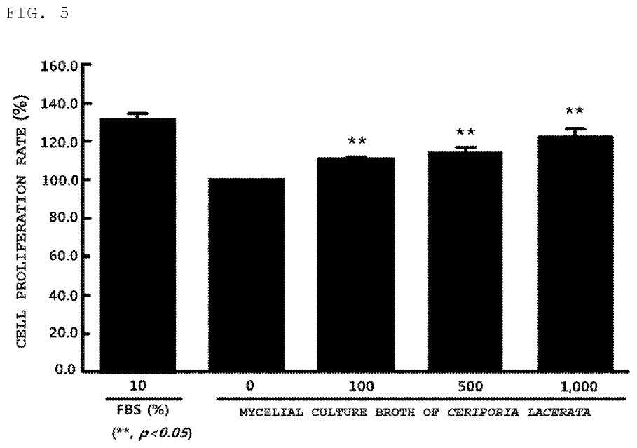

Furthermore, HaCaT cells wounded at the center thereof were treated with the mycelial culture broth of Ceriporia lacerata, and then cultured for 12 hours, 48 hours, and 72 hours, and changes in wound size according to the concentration and culture time of the mycelial culture broth were observed. As a result, it was confirmed that the wound size was significantly reduced in the groups treated with the mycelial culture broth of Ceriporia lacerata (see FIG. 4).

In addition, HaCaT cells were treated with the mycelial culture broth of Ceriporia lacerata, and then cultured for 12 hours, 48 hours, and 72 hours, and a cell proliferation rate according to culture time was measured. As a result, it was confirmed that the cell proliferation rate was increased in the groups treated with the mycelial culture broth of Ceriporia lacerata (see FIG. 5).

From these results, it was confirmed that a pharmaceutical composition for wound healing including the mycelial culture broth of Ceriporia lacerata increased the proliferation rate of cells and promoted wound recovery. Therefore, the pharmaceutical composition of the present invention may be effectively used for wound healing.

The mycelial culture broth or an extract thereof may be included in an amount of 0.1 wt % to 80 wt % or 0.1 wt % to 50 wt % with respect to a total weight of the pharmaceutical composition for wound healing. An effective amount of the mycelial culture broth or an extract thereof may be appropriately adjusted according to the use method and purpose of the pharmaceutical composition.

The pharmaceutical composition may include, as an active ingredient, a mycelial culture broth of Ceriporia lacerata, dry powder of the mycelial culture broth, or an extract of the mycelial culture broth.

The pharmaceutical composition may further include a suitable carrier, excipient, and diluent which are commonly used.

The pharmaceutical composition may be formulated using conventional methods and used. Examples of suitable formulations include, but are not limited to, tablets, pills, powders, granules, sugar-coated tablets, hard or soft capsules, solutions, suspensions or emulsions, injections, and suppositories.

The pharmaceutical composition may be prepared into a suitable formulation using a pharmaceutically inert organic or inorganic carrier. That is, when the formulation is in the form of tablets, coated tablets, sugar-coated tablets, and hard capsules, the organic or inorganic carrier may include lactose, sucrose, starch or a derivative thereof, talc, calcium carbonate, gelatin, or stearic acid or a salt thereof. In addition, when the formulation is in the form of soft capsules, the organic or inorganic carrier may include vegetable oil, wax, fat, and semisolid and liquid polyol. Furthermore, when the formulation is in the form of a solution or syrup, the organic or inorganic carrier may include water, polyol, glycerol, and/or vegetable oil.

The pharmaceutical composition may further include, in addition to the above-described carriers, a preservative, a stabilizer, a wetting agent, an emulsifier, a solubilizing agent, a sweetener, a colorant, an osmotic pressure control agent, an antioxidant, and the like.

The administration method of the pharmaceutical composition may be easily selected according to a dosage form, and the pharmaceutical composition may be administered orally or parenterally. A dosage may vary depending on the age, gender, and body weight of a patient, the severity of disease, and administration route, and generally, the pharmaceutical composition may be administered in an amount of 5 mg/kg to 1,000 mg/kg, particularly, 10 mg/kg to 600 mg/kg, with respect to the amount of the mycelial culture broth, which is the active ingredient, once to three times a day. However, the dosage is not intended to limit the scope of the present invention.

The administration form of the pharmaceutical composition may be, but is not limited to, topical application or intradermal injection to a site in need of wound healing.

Another embodiment of the present invention provides a cosmetic composition for wound healing including, as an active ingredient, a mycelial culture broth of Ceriporia lacerata or an extract thereof.

The cosmetic composition may include, as an active ingredient, a mycelial culture broth of Ceriporia lacerata, dry powder of the mycelial culture broth, or an extract of the mycelial culture broth.

The cosmetic composition may be formulated into a cosmetic formulation commonly prepared in the art. The cosmetic composition may be formulated into, for example, but not limited to, a solution, a suspension, an emulsion, a paste, a gel, a cream, a lotion, a powder, a powder foundation, an emulsion foundation, a wax foundation, and a spray. More specifically, the cosmetic composition may be formulated in the form of a skin lotion, a nourishing lotion, a nourishing cream, a massage cream, an essence, an eye cream, a pack, a spray, or a powder.

Another embodiment of the present invention provides a use of a mycelial culture broth of Ceriporia lacerata or an extract thereof for healing a skin wound.

Another embodiment of the present invention provides a use of a mycelial culture broth of Ceriporia lacerata or an extract thereof for preparing a drug for healing a skin wound.

Another embodiment of the present invention provides a method of healing a skin wound, including administering, to a subject in need thereof, a mycelial culture broth of Ceriporia lacerata or an extract thereof.

The subject may be a mammalian animal, more particularly, a human.

The mycelial culture broth of Ceriporia lacerata, dry powder of the mycelial culture broth, or an extract of the mycelial culture broth are the same as described above.

### Mode

Hereinafter, the present invention will be described in further detail with reference to the following examples. However, these examples are provided for illustrative purposes only and are not intended to limit the scope of the present invention.

### Preparation Example 1. Preparation of Mycelial Culture Broth of Ceriporia lacerata

1.1. Preparation of Pre-Fermentation Medium

A pre-fermentation medium was prepared in an amount of 0.1% of a main-fermentation medium, and the medium used upon pre-fermentation is a potato dextrose broth (PDB, 254920, Difco, USA). The PDB medium was dissolved in distilled water at a concentration of 24 g/l in a 1 L Erlenmeyer flask according to the manufacturer's instructions, and pH of the dissolved medium was 4.5±0.1. A hole in the Erlenmeyer flask was sealed with a stopper and then sealed once again with aluminum foil. Subsequently, the resulting solution was subjected to high-temperature wet sterilization at 121° C. for 15 minutes, and the sterilized pre-fermentation medium was left for 6 hours on a clean bench (HB-402V, HANBAEK, Korea) to lower the temperature to room temperature.

1.2. Pre-Fermentation

A parent strain grown through subculture of Ceriporia lacerata was freeze-stored at −80° C., and the freeze-stored strain was sub-cultured twice or three times in a potato dextrose agar (PDA) medium (87 plastic bulbs; Difco, Becton Dickinson and Company) (hereinafter referred to as “PDA culture strain”) and stored in a refrigerator at 4° C. until use.

1 PDA culture strain and 200 ml of the pre-fermentation medium of Preparation Example 1.1 were added to a mixer sterilized on a clean bench and mixed for 80 seconds, and then 400 ml of the pre-fermentation medium of Preparation Example 1.1 was added thereto and mixed. Thereafter, the mixed solution was added to a new Erlenmeyer flask and sealed with a stopper, followed by incubation for 9 days in a shaking incubator (HB-201SL, HANBAEK, Korea) set at 25° C. and 150 rpm.

1.3. Preparation of Main-Fermentation Medium

The raw materials shown in Table 1 below and distilled water were placed in a medium dissolution tank, and an impeller was operated for 15 minutes to sufficiently dissolve the medium. Distilled water was added to the dissolved medium so that the total amount reached 600 L and the concentration of each raw material became the concentration shown in Table 1 below. Thereafter, the medium was sterilized at 121° C. for 60±5 minutes using a production fermentor (Aerobic type, Non-stirred).

TABLE 1

| Raw material | Concentration | |
|---|---|---|
| Glucose | 12.5 | g/l |
| Potato starch | 2.5 | g/l |
| Defatted soybean | 5.0 | g/l |
| Silicon-containing anti-foaming agent (Product name: | 0.125 | l |
| TJ2002) | | |

1.4. Main Fermentation

The sterilized medium of Preparation Example 1.3 was cooled to a temperature of 22±1° C. using cooling water, and filtered air was continuously injected to prevent negative pressure, thereby preventing external contamination. A flame was generated around an inoculation port to prevent the inflow of foreign particles, a flask was placed inside the flame, and seeds were inoculated into the flask. After completion of the inoculation, the medium was cultured for about 11 days while adjusting the air inflow from 0.05 vvm to 1 vvm (volume/medium volume·minute) to prepare a mycelial culture broth of Ceriporia lacerata.

1.5. Preparation of Ceriporia lacerata Culture Broth Powder

The mycelial culture broth of Ceriporia lacerata of Preparation Example 1.4 was lyophilized in vacuum using a vacuum freeze-dryer at 25° C. for 72 hours and powdered, thereby preparing mycelial culture broth powder of Ceriporia lacerata.

### Preparation Example 2. Extract of Mycelial Culture Broth of Ceriporia lacerata

900 ml of ethyl acetate was added to 600 ml of the mycelial culture broth of Ceriporia lacerata prepared according to Preparation Example 1.4 and mixed, and then left. Subsequently, the ethyl acetate layer was separated from the left material, and then concentrated under reduced pressure using a rotary evaporator at 35° C. The concentrate obtained under reduced pressure was freeze-dried in vacuum using a vacuum freeze-dryer at 25° C. for 72 hours to obtain an ethyl acetate extract of the mycelial culture broth of Ceriporia lacerata.

### Experimental Example 1. Evaluation of Wound Healing Capacity of HT-29 Cells Upon Treatment with Mycelial Culture Broth of Ceriporia lacerata

A human colorectal adenocarcinoma cell line (HT-29) received from the Korea Cell Line Bank was cultured in RPMI 1640 medium containing 10% fetal bovine serum (FBS) and 1% penicillin-streptomycin (P/S) for 48 hours. Subsequently, the cells were inoculated into a 12-well plate for cell culture at a density of 3×10³ cells/well and cultured. Thereafter, when the cells were attached, the center of a portion where the cells were cultured was scratched with a micropipette tip, and then an image corresponding to 0 hours was taken. Then, the mycelial culture broth powder of Ceriporia lacerata prepared according to Preparation Example 1.5 was dissolved in DMSO, the cells were treated with the resulting solution at various concentrations (0 μg/ml, 100 μg/ml, 500 μg/ml, and 1,000 μg/ml) and incubated in a CO₂ incubator for 12 hours, 48 hours, and 72 hours to observe a change in wound size according to time.

As illustrated in FIGS. 1 and 2, the wound size was reduced in the groups treated with the mycelial culture broth of Ceriporia lacerata, compared to a control (0 μg/ml). In particular, the biggest reduction in wound size over time was observed in the experimental group treated with 500 μg/ml of the mycelial culture broth of Ceriporia lacerata.

### Experimental Example 2. Evaluation of Proliferation Rate of C2C12 Cells Upon Treatment with Mycelial Culture Broth of Ceriporia lacerata

A mouse myoblast cell line (C2C12) received from the Korea Cell Line Bank was cultured in DMEM containing 10% FBS and 1% P/S for 48 hours. Subsequently, the cells were inoculated into a 24-well plate for cell culture at a density of 1.5×10³ cells/well and cultured. Thereafter, when the cells were attached, the mycelial culture broth powder of Ceriporia lacerata prepared according to Preparation Example 1.5 was dissolved in DMSO, and the cells were treated with the resulting solution according to various concentrations (0 μg/ml, 100 μg/ml, 500 μg/ml, and 1,000 μg/ml) and incubated in a CO₂ incubator for 24 hours. 100 pl of the culture broth of the cultured C2C12 cells was dispensed into each well of a 96-well plate, and then absorbance at a wavelength of 450 nm was measured using a CCK-8 assay kit (Dojindo, USA) to determine the cell proliferation rate of each group.

As illustrated in FIG. 3, the cell proliferation rate was increased in the groups treated with the mycelial culture broth of Ceriporia lacerata, compared to the control (0 μg/ml). In particular, the highest cell proliferation rate was shown in the group treated with 500 μg/ml of the mycelial culture broth of Ceriporia lacerata.

### Experimental Example 3. Evaluation of Wound Recovery Capacity of HaCaT Cells Upon Treatment with Mycelial Culture Broth of Ceriporia lacerata

Human keratinocytes (HaCaT) received from the CLS cell line service (Germany) were cultured in RPMI 1640 medium containing 10% FBS and 1% P/S for 48 hours. Subsequently, the cells were inoculated into a 12-well plate for cell culture at a density of 3×10³ cells/well and cultured. Thereafter, when the cells were attached, the center of a portion where the cells were cultured was scratched with a micropipette tip, and then an image corresponding to 0 hours was photographed. Then, the mycelial culture broth powder of Ceriporia lacerata prepared according to Preparation Example 1.5 was dissolved in DMSO, and the cells were treated with the resulting solution at various concentrations (0 μg/ml, 100 μg/ml, 500 μg/ml, and 1,000 μg/ml) and incubated in a CO₂ incubator for 12 hours, 48 hours, and 72 hours to observe a change in wound size according to time.

As illustrated in FIG. 4, the wound size was reduced in the groups treated with the mycelial culture broth of Ceriporia lacerata, compared to the control (0 μg/ml). In particular, the biggest reduction in wound size over time was observed in the experimental group treated with 1,000 μg/ml of the mycelial culture broth of Ceriporia lacerata.

### Experimental Example 4. Evaluation of HaCaT Cell Proliferation Rate Upon Treatment with Mycelial Culture Broth of Ceriporia lacerata

A HaCaT cell line received from the Korea Cell Line Bank was cultured in DMEM containing 10% FBS and 1% P/S for 48 hours. Subsequently, the cells were inoculated into a 24-well plate for cell culture at a density of 1.5×10³ cells/well and cultured. Thereafter, when the cells were attached, the mycelial culture broth powder of Ceriporia lacerata prepared according to Preparation Example 1.5 was dissolved in DMSO, and the cells were treated with the resulting solution according to various concentrations (0 μg/ml, 100 μg/ml, 500 μg/ml, and 1,000 μg/ml) and incubated in a CO₂ incubator for 24 hours. 100 μl of the culture broth of the cultured HaCaT cells was dispensed into each well of a 96-well plate, and then absorbance at a wavelength of 450 nm was measured using a CCK-8 assay kit to determine the cell proliferation rate of each group.

As illustrated in FIG. 5, the cell proliferation rate was increased in the groups treated with the mycelial culture broth of Ceriporia lacerata compared to the control (0 μg/ml), and specifically, the highest cell proliferation rate was shown in the group treated with 1,000 μg/ml of the mycelial culture broth of Ceriporia lacerata.

As confirmed from the results of Experimental Examples 1 to 4, it was confirmed that the mycelial culture broth of Ceriporia lacerata increased a cell proliferation rate and promoted wound healing. An extract of the mycelial culture broth also exhibited the same effects.

1. A pharmaceutical composition for wound healing, the pharmaceutical composition comprising, as an active ingredient, a mycelial culture broth of Ceriporia lacerata or an extract thereof.

2. The pharmaceutical composition according to claim 1, wherein the mycelial culture broth is obtained through culturing at a temperature of 15° C. to 30° C. for 3 days or longer.

3. The pharmaceutical composition according to claim 1, wherein the mycelial culture broth is obtained through culturing at a temperature of 15° C. to 30° C. for 9 days or longer.

4. The pharmaceutical composition according to claim 1, wherein the mycelial culture broth is obtained through culturing at a temperature of 15° C. to 30° C. for 11 days or longer.

5. The pharmaceutical composition according to claim 1, wherein the extract is obtained through extraction of the mycelial culture broth of Ceriporia lacerata with a solvent selected from the group consisting of distilled water, C₁ to C₄ lower alcohol, acetone, ether, chloroform, and ethyl acetate, or a mixture thereof.

6. The pharmaceutical composition according to claim 1, wherein the mycelial culture broth or an extract thereof is included in an amount of 0.1 wt % to 80 wt % with respect to a total weight of the pharmaceutical composition.

7. The pharmaceutical composition according to claim 1, wherein the mycelial culture broth is obtained by culturing a mycelium of Ceriporia lacerata in a medium containing glucose.

8. A cosmetic composition for ameliorating wound healing, the cosmetic composition comprising, as an active ingredient, a mycelial culture broth of Ceriporia lacerata or an extract thereof.

9. A use of a mycelial culture broth of Ceriporia lacerata or an extract thereof for healing a skin wound.

10. A use of a mycelial culture broth of Ceriporia lacerata or an extract thereof for preparing a drug for healing a skin wound.

11. A method of healing a skin wound, the method comprising administering, to a subject in need thereof, a mycelial culture broth of Ceriporia lacerata or an extract thereof.
